(19) United States
(12) Patent Application Publication
(10) Pub. No.: US 2008/0287939 A1
(43) Pub. Date: Nov. 20, 2008

(54) ENDOVASCULAR THERMAL TREATMENT DEVICE WITH FLEXIBLE GUIDE TIP AND METHOD

(76) Inventors: William M. Appling, Granville, NY (US); Ralph A. MEYER, Argyle, NY (US); Leonard G. Schaefer, Queensbury, NY (US)

Correspondence Address: AFS / ANGIODYNAMICS, 666 THIRD AVENUE, FLOOR 10, NEW YORK, NY, 10017, US

(21) Appl. No.: 12/164,674

(22) Filed: Jun. 30, 2008

Related U.S. Application Data
(63) Continuation-in-part of application No. 11/777,198, filed on Jul. 12, 2007.
Application No. 11/777,198 is a continuation of application No. 10/613,395, filed on Jul. 3, 2003, now Pat. No. 7,273,478.
(60) Provisional application No. 60/395,218, filed on Jul. 10, 2002.
(60) Provisional application No. 60/948,878, filed on Jul. 10, 2007.
(60) Provisional application No. 60/948,226, filed on Jul. 6, 2007.

(51) Int. Cl. A61B 18/24; A61B 18/18

(52) U.S. Cl. 606/15; 606/33

(57) ABSTRACT

An elongated thermal energy delivery device for use in endovenous thermal treatment of blood vessel is provided. The energy device includes a flexible guide tip attached to its distal portion which provides for direct tracking and advancement of the energy device through the vein without the use of a treatment sheath. Also provided is a method of using the thermal energy delivery device with flexible guide tip. The method eliminates the need for a treatment sheath and accessory procedural components and the procedural steps associated with these components.

### CROSS REFERENCE TO RELATED APPLICATIONS

This is a continuation-in-part application of Ser. No. 11/777,198, filed Jul. 12, 2007, which is a continuation of Ser. No. 10/613,395, filed Jul. 3, 2003, which claims priority under 35 U.S.C. §119(e) to U.S. Provisional application Ser. No. 60/395,218, filed Jul. 10, 2002, all of which are incorporated herein by reference.

This application also claims priority under 35 U.S.C. §119(e) to U.S. Provisional application Ser. No. 60/948,878, filed Jul. 10, 2007 and U.S. Provisional application Ser. No. 60/948,226, filed Jul. 6, 2007, all of which are incorporated herein by reference.

### FIELD OF THE INVENTION

The present invention relates to a medical apparatus and method for treatment of blood vessels. More particularly, the present invention relates to an endovascular apparatus and method for minimally invasive treatment of venous reflux disease.

### BACKGROUND OF THE INVENTION

Veins can be broadly divided into three categories: the deep veins, which are the primary conduit for blood return to the heart; the superficial veins, which parallel the deep veins and function as a channel for blood passing from superficial structures to the deep system; and topical or cutaneous veins, which carry blood from the end organs (e.g., skin) to the superficial system. Veins have thin walls and contain one-way valves that control blood flow. Normally, the valves open to allow blood to flow into the deep veins and close to prevent back-flow into the superficial veins. When the valves are malfunctioning or only partially functioning, however, they no longer prevent the back-flow of blood into the superficial veins. This condition is called reflux. As a result of reflux, venous pressure builds within the superficial system. This pressure is transmitted to topical veins, which, because the veins are thin walled and not able to withstand the increased pressure, become dilated, tortuous or engorged.

In particular, venous reflux in the lower extremities is one of the most common medical conditions of the adult population. It is estimated that venous reflux disease affects approximately 25% of adult females and 10% of adult males. Symptoms of reflux include varicose veins and other cosmetic deformities, as well as aching and swelling of the legs. Varicose veins are common in the superficial veins of the legs, which are subject to high pressure when standing. Aside from being cosmetically undesirable, varicose veins are often painful, especially when standing or walking. If left untreated, venous reflux may cause severe medical complications such as bleeding, phlebitis, ulcerations, thrombi and lipodermatosclerosis (LDS).

When veins become enlarged, the leaflets of the valves no longer meet properly. Blood collects in the superficial veins, which become even more enlarged. Since most of the blood in the legs is returned by the deep veins, and the superficial veins only return about 10%, they can be removed or closed down without serious harm. Endovascular thermal therapy is a minimally invasive treatment involving the delivery of thermal energy generated by laser, or radio or microwave frequencies, to cause vessel occlusion or ablation. Thermal energy is delivered to the vein wall or blood (depending on the device and method of treatment) using an energy source that is placed within the vein and withdrawn while the energy is emitted. The device and method of treatment can vary significantly depending on the type of energy used. For example, devices that employ laser energy involve inserting a fiber optic line into the vein to deliver laser energy to the blood within the vein to heat the blood and, in turn, heat the walls of the vein. Contact between the emitting face of the fiber and the vein wall is typically avoided in order to prevent perforating the vein and the pain and bruising associated with such perforations. In RF devices, on the other hand, a device with electrodes is inserted into the vein. In order for such devices to work, and in contrast to laser devices, the electrodes should be placed into contact with the vein wall and maintained in contact throughout the delivery of the RF energy. Thus, RF devices are significantly different than laser devices, and the associated methods involve different steps.

Current endovenous treatment using either laser or RF energy requires numerous steps and medical components. A typical laser procedure involves the following steps. First, the vein is accessed using a small gauge needle. An 0.018″ guidewire is inserted into the lumen of the needle and advanced into the vein. Once access is gained, the needle is removed and a micropuncture dilator/sheath set is advanced over the guidewire and into the vein. Typically, the dilator/sheath set is a 5F size in order to allow insertion of a 0.035″ procedure guidewire. The dilator is removed and the larger guidewire is inserted into the vein. The micropuncture dilator is then removed, leaving just the 0.035″ guidewire in place. A longer, larger treatment sheath with dilator is then threaded over the guidewire into the vein. After removing the treatment dilator and guidewire, the fiber is inserted into the treatment sheath and advanced until the fiber face is flush with the distal end of the sheath. The sheath is then retracted so as to expose the distal section of the fiber. In some devices there is a mechanism for locking the retracted sheath to the fiber at the proximal hub to stabilize the fiber position relative to the sheath. Once both the fiber and sheath are positioned, the user administers tumescent anesthesia along the vein. If necessary, the fiber tip position may be adjusted after tumescent anesthesia delivery. The last step of the procedure is to pull back the fiber/sheath through the vein while energy is emitted from the emitting face at the tip of the fiber.

A typical procedure takes between 45 minutes to 90 minutes, depending on the patient's anatomy, length of the treatment vein and other procedural factors. Of the total procedure time, only between about 3 and 7 minutes is devoted to the actual application of laser energy within the vein. The majority of the procedure time is devoted to accessing the vein, placing the fiber, and administering tumescent anesthesia.

Therefore, it would be desirable to provide an endovascular treatment device and method which reduces the number of procedural steps required to complete the treatment. Eliminating individual procedural steps may reduce the overall procedure time, thereby reducing physician costs. Reducing procedure time by eliminating specific steps also may contribute to reducing complication rates. Costs associated with the medical components no longer required also may be reduced or eliminated.

### SUMMARY OF THE DISCLOSURE

In accordance with a first aspect, the present invention is directed to an endovascular thermal treatment device. The device comprises an elongated, thermal energy delivery device having at its distal portion an energy emitting section, and a flexible guide tip attached to the distal portion of the energy delivery device and extending distally therefrom, the flexible guide tip adapted to guide the energy emitting section through a blood vessel.

In some embodiments of the present invention, the energy delivery device includes an elongated optical fiber having at its distal end an energy emitting face for emitting laser energy therefrom. In other embodiments of the present invention, the energy delivery device includes at least one electrode for delivering RF or other electrical energy. In some embodiments of the present invention, the energy application device further includes a shield disposed annularly about the energy emitting section and extending distally therefrom. Preferably, the shield includes one or more windows for permitting the flow of blood therethrough. The flexible guide tip is preferably attached to a distal portion of the shield to facilitate insertion and/or advancement through the blood vessel.

In accordance with another aspect, the present invention is directed to a an endovascular treatment method for causing closure or reducing the diameter of a blood vessel comprising the following steps:

advancing into a blood vessel an elongated thermal energy delivery device having at its distal portion an energy emitting section, the distal portion being attached to a flexible guide tip that extends distally from the distal portion;

applying thermal energy through the energy emitting section while longitudinally moving the advanced energy delivery device.

One advantage of the device of the present invention is that it can allow for the elimination of many of the steps required in prior art treatment methods. The shield protects the energy emitting face of the optical fiber, for example, and further prevents the possibility of inadvertent contact between the vessel wall and fiber tip, and any associated vessel perforations. Yet another advantage of the device of the present invention is that the flexible guide tip facilitates insertion and/or advancement of the device without a procedure sheath/dilator set, thereby reducing the number of procedure steps and kit components required for the treatment. In some currently preferred embodiments of the present invention, the ultrasonically visible braiding or other structure on the device can provide a highly visible target to guide the injection of tumescent anesthesia along the vein segment to be treated. The braiding or other structure also can provide additional protection against damage to the device, such as to a fiber shaft.

Other objects and advantages of the apparatus and method of the present invention will become more readily apparent in view of the following detailed description of the currently preferred embodiments and accompanying drawings.

### BRIEF DESCRIPTION OF THE DRAWINGS

FIG. 1 is a plan view of a laser fiber with flexible guide tip of the present invention.

FIGS. 2A and 2B are enlarged partial cross-sectional views of the distal section of the device taken along line A-A of FIG. 1.

FIG. 3 is a cross-sectional view and end view of the shield-to-flexible guide tip connector of the present invention.

FIGS. 4A and 4B are enlarged cross-sectional views of two embodiments of a reinforced fiber shaft of the present invention.

FIGS. 5A and 5B are flowcharts illustrating methods of endovenous treatment. FIG. 5A depicts a prior art method of treatment. FIG. 5B illustrates an improved method in accordance with the present invention.

FIGS. 6A and 6B are enlarged partial cross-sectional views of the distal sections of additional embodiments of an optical fiber with flexible guide tip of the present invention.

FIGS. 7A and 7B are enlarged perspective views of two embodiments of a shield of the present invention.

FIG. 8 is a partially plan view of a radiofrequency catheter with flexible guide tip of the present invention.

FIG. 9 is a partially plan view of a microwave catheter with flexible guide tip of the present invention.

### DETAILED DESCRIPTION OF THE INVENTION

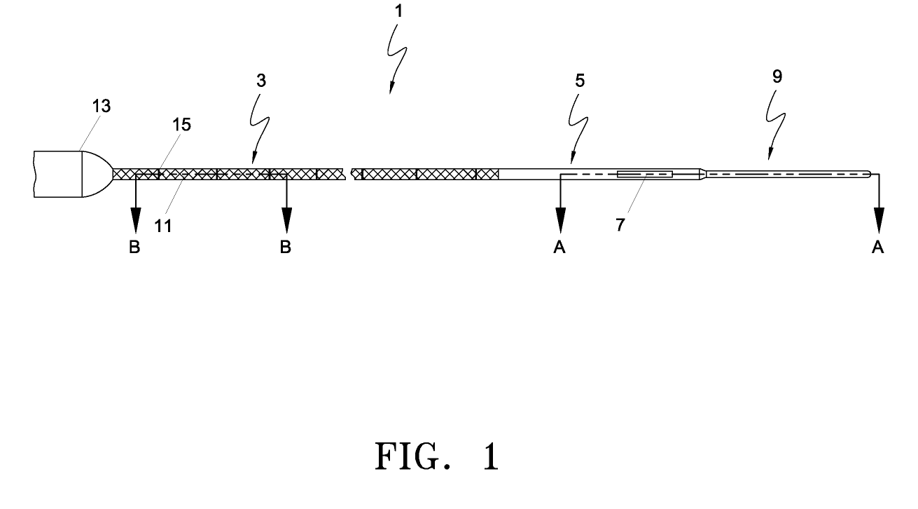

One embodiment of the present invention is illustrated in FIGS. 1 through 7. An endovascular optical fiber with flexible guide tip 1 is illustrated in FIG. 1. The fiber with flexible guide tip 1 includes a proximal SMA connector 13, a reinforced fiber shaft 3, a shield 5, and a flexible guide tip 9. The term “guide tip” is used herein to mean a flexible member used to introduce and guide an intravascular device, or similar structure that is currently known, or that later becomes known for performing this function. In one embodiment, the flexible guide tip 9 is the tip of a guide wire which is used to access a blood vessel. One end of the SMA connector 13 is adapted to connect with a laser (not shown) and the other end is attached to the reinforced fiber shaft 3. The reinforced fiber shaft 3 is comprised of a fiber optic line coaxially surrounded by a protective outer layer containing ultrasonically visible strands or reinforcement members 11. The fiber shaft 3 can include graduated markings 15. Attached to the distal portion of the reinforced fiber shaft is the shield 5 which includes at least one window 7 in the sidewall of the shield. Attached to the distal portion of the shield 5 is the flexible guide tip 9 which extends in a distal direction for approximately 2 to 2.5 centimeters.

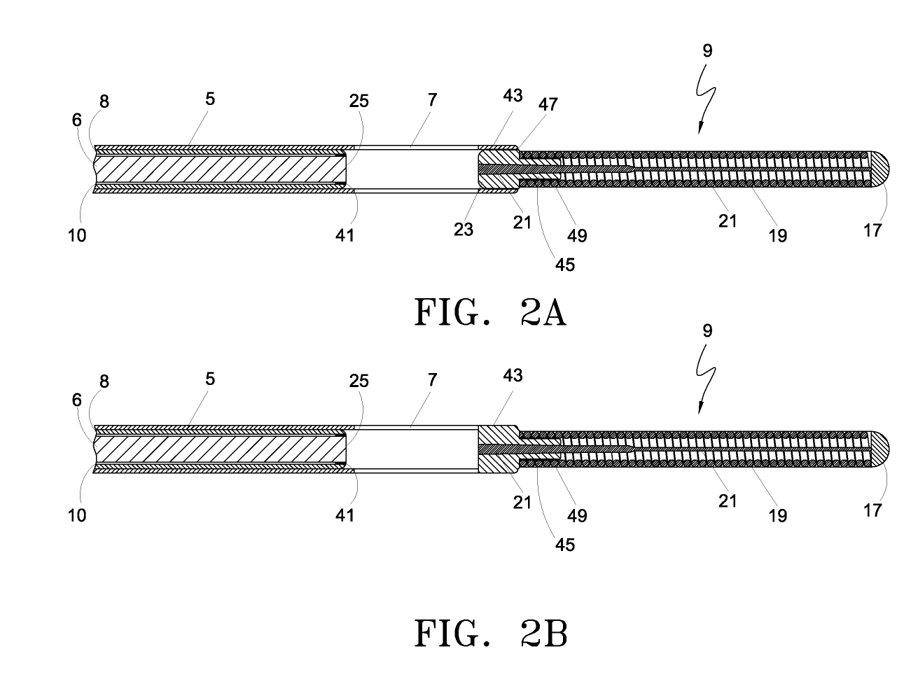

FIG. 2A illustrates an enlarged cross-sectional view of the assembled distal portion of the optical fiber with flexible guide tip 1 taken along line A-A of FIG. 1. The distal portion is comprised of an energy emitting face 25 of the optical fiber 6, shield 5, shield-to-flexible guide tip connector 23 and flexible guide tip 9. The fiber includes a wavelength transmitting core 6 coaxially surrounded by a silica layer 8. An annular space 10 between the transmitting core and silica layer 8 creates an air gap 10 which terminates at a weld point 41. Emitting face 25 of optical fiber core 6 transmits laser energy from the core in a forward facing direction, as disclosed in provisional patent application No. 60/913,767, which is incorporated herein by reference.

In the illustrated embodiment, shield 5 is disposed annularly about the emitting face 25 of the fiber 6 and extends distally therefrom, and is preferably comprised of a metallic material such as stainless steel or titanium. The shield 5 can be coated with gold, titanium nitride, or other material or structure that is currently known, or that later becomes known, to further enhance visibility under ultrasound. In the illustrated embodiment, the shield 5 is preferably about 0.63″ in length with an outer diameter of about 0.05″ and an inner diameter of about 0.042″. The shield 5 is coaxially arranged around the laser fiber distal end as shown in FIG. 2A. The energy emitting face 25 of the fiber is positioned proximal to but not flush with the at least one window 7 edge in the shield 5. The shield 5 further prevents the possibility of the front energy emitting face 25 from inadvertently coming into contact with the vessel wall, and thereby further prevents the possibility of inadvertent perforations and subsequent patient discomfort and bruising.

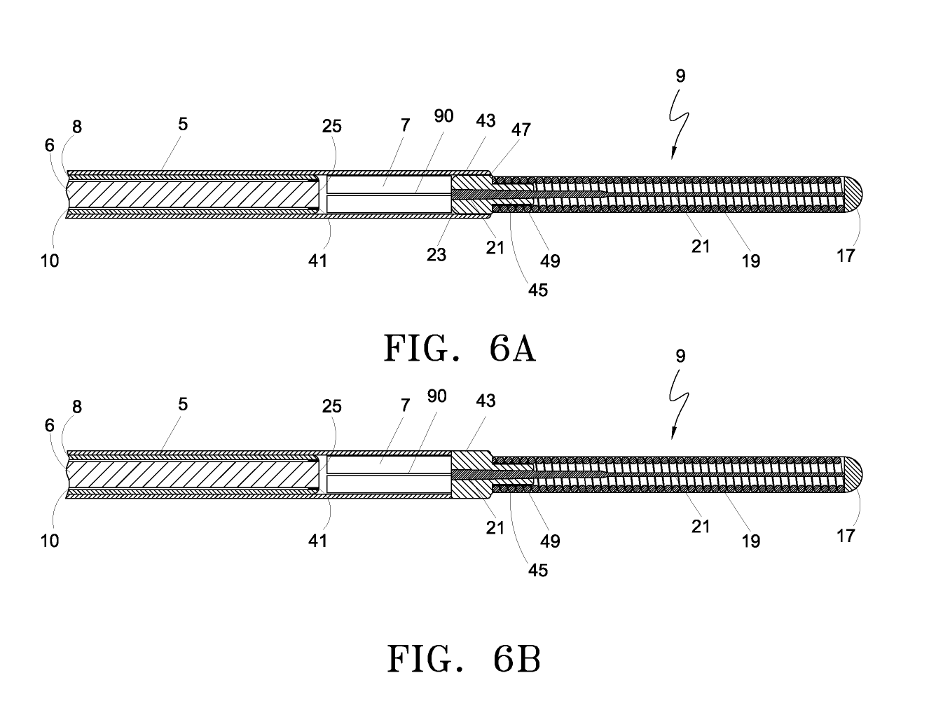

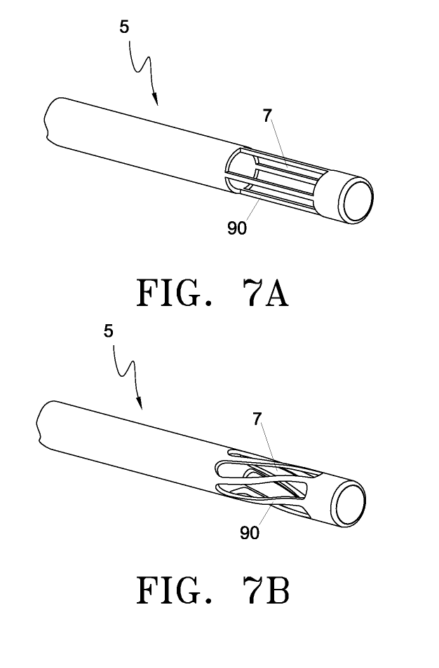

When the laser is activated, laser energy is directed in a forward fashion through the core 6 exiting from the energy emitting face 25. The windows 7 of shield 5 permit blood flow therethrough and into the laser emitting path to facilitate absorption of the laser energy by the blood, and the resultant conversion of the laser energy into thermal energy to substantially uniformly heat the surrounding vessel wall. In the illustrated embodiment, the shield 5 defines a plurality of windows 7, wherein each window is defined by an axially extending aperture formed through the shield 5. Also in the illustrated embodiment, there are two windows 7 being located on diametrically opposite sides of the shield 5 relative to each other, with a length of approximately 0.12″ and width of approximately 0.052″Distal end segment 43 of shield 5 extends from the distal end of each window 7 distally for approximately 0.04″As may be recognized by those of ordinary skill in the pertinent art based on the teachings herein, the number of windows, and the dimensions of the windows and other features of the device disclosed herein, are only exemplary, and numerous other variations of such features and dimensions equally may be employed. See, for example, the discussion related to FIGS. 6A, 6B, 7A, 7B, infra.

Still referring to FIG. 2A, flexible guide tip 9 is comprised of distal end rounded portion such as a weld ball 17, a compression spring or coil 19, and a mandrel wire 21, as is well known in the art. In the illustrated embodiment, the overall length of the flexible guide tip 9 is approximately 2 centimeters; the coil 19 and distal end weld ball 17 define an outer diameter of approximately 0.035″ and mandrel wire 21 is approximately 0.006″ in diameter at its proximal end transitioning to a flattened approximately 0.003″ wire for the distal portion of about 2 cm. When assembled, mandrel wire 21 extends longitudinally through the flexible guide tip 9 and into the lumen 27 of connector 23. Mandrel wire 21 provides a structural backbone to prevent the turns of the coil 19 from separating and further ensures that the flexible guide tip 9 and connector 23 are fixedly secured.

One advantage of this construction is that it provides the flexibility and pushability of the flexible guide tip 9 necessary to track and advance the fiber 6 through the vasculature. Another advantage of a flexible guide tip design is that it eliminates the need for a separate guidewire and procedure sheath/dilator set. In yet another aspect of the invention, the guide tip is dimensioned so that when the distal end weld ball 17, which is ultrasonically visible, is positioned relative to the sapheno-femoral junction, the energy emitting face 25 of the fiber 6 is located approximately at a desired start position for treatment. Due to the relatively high mass density, the weld ball 17 is more ultrasonically visible than the remaining portion of the guide tip 9.

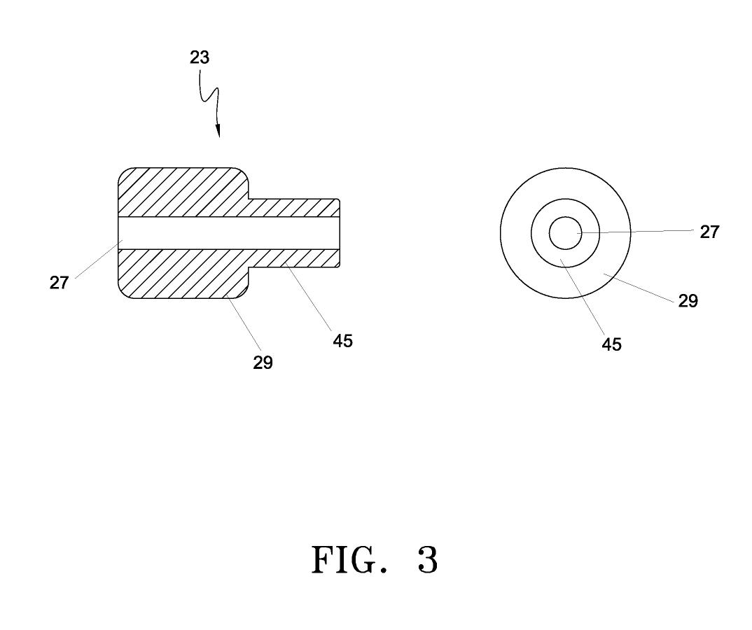

Referring to FIG. 3, the shield-to-flexible guide tip connector 23 is illustrated in cross-section and as an end view. Connector 23 is comprised of a base portion 29 and an extension portion 45. A through lumen 27 extends longitudinally through the base 29 and extension 45. Referring back to FIG. 2A, the base portion 29 of connector 23 is positioned within the distal opening of end segment 43 of shield 5. Connector 23 is permanently attached to the shield 5 by a weld 47 along the outer surface of base 29 or by other attachment mechanisms known in the art. Extension 45 of connector 23 extends distally into the coil of flexible guide tip 9. Extension 45 is welded to coil 19 at axially-extending area 49 or otherwise is attached to the inside surface of coil 19 in a manner known to those of ordinary skill in the pertinent art.

FIG. 2B illustrates an alternative embodiment of the assembled distal portion of the laser fiber with flexible guide tip 1 taken along line A-A of FIG. 1. In this embodiment the shield 5 includes distal end segment 43 as a unitary structure. Distal end segment 43 is a solid structure including an extension section 45 and contains a through lumen into which the mandrel wire 21 of the flexible guide tip 9 is inserted. One advantage of this embodiment is that the weld joint 47 is eliminated, thereby enabling an increase in the overall structural integrity of the device.

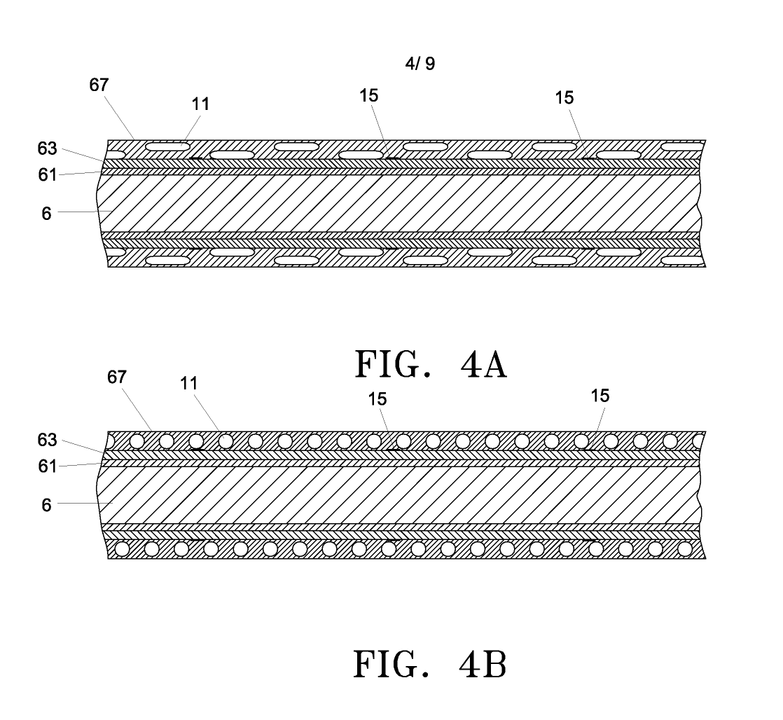

FIG. 4A and FIG. 4B illustrate enlarged longitudinal cross-sectional views of the fiber shaft 3 taken along line B-B of FIG. 1. In FIG. 4A, the fiber shaft 3 includes a solid inner core 6 enclosed in a cladding material 61 and further surrounded by a protective jacket 63, also known as a buffer. The fiber 6 is further protected by an outer layer comprised of a series of reinforcement members 11. The reinforcement members may be in the form of a metallic braid, weave or coil, preferably of medical grade stainless steel or nitinol. FIG. 4A depicts a reinforcement pattern representing a braided weave. FIG. 4B represents a coiled reinforcement pattern 11. Standard braiding or coiling machines can be used to apply the various reinforcement patterns directly over the fiber shaft. For example, a braid pattern of 32 pics per inch using approximately 0.002″ diameter 304 stainless steel wire may be used.

The reinforcement members 11 are embedded in a polymer overlay 67 as shown in FIGS. 4A and 4B. Compression from the braiding combined with the polymer overlay 67 ensures that the reinforcement members 11 remain in position on the shaft 3 surface. The polymer overlay 67 provides a smooth outer surface finish to the shaft 3 to facilitate advancement through the vessel. The overlay 67 can be either extruded directly onto the shaft 3 surface over the reinforcement layer 11 or can be applied using a shrink tubing process. Possible materials include but are not limited to PTFE, FEP, PET or PEBAX. Alternatively, the matrix 67 can be applied by a spray coating process. The overlay 67 is preferably translucent to allow visibility of the graduated shaft markings 15 on the buffer 63 surface. Alternatively, the graduated markings 15 can be applied directly to the polymer overlay 67. A lubricous coating may be applied to the finished laser shaft 3 and the flexible guide tip to further enhance ease of advancement through the vessel.

The reinforcement member 11 pattern increases the diameter of the bare fiber shaft 3 which in the illustrated embodiment is by approximately 0.008″. Also in the illustrated embodiment, the increase in diameter of the shaft 3 due to the addition of the polymer overlay 67 is between approximately 0.004-0.012″ depending upon the material and application process. For a 600 micrometer fiber which has an outer diameter of 0.041″, the outer diameter of the finished reinforced laser fiber 3 is approximately 0.05″. This diameter corresponds with the approximate outer diameter of the shield 5, providing a smooth transition between the shaft section 3 and the distal end segment of the laser fiber with flexible guide tip 1.

As an alternative embodiment of the reinforced fiber shaft, the reinforcement pattern can be designed to replace the shield 5. In this embodiment the reinforcement members 11 coaxially surround the fiber shaft 3, extending beyond the distal end (or energy emitting face) of the fiber 6 to connect with the flexible guide tip 9. The windows 7 are formed by customizing the braid pattern into a series of longitudinal bundles with gaps between. The braid bundles are of sufficient strength to maintain the gap distance between the energy emitting face 25 of the fiber and the proximal section of the flexible guide tip 9.

Other methods of increasing the ultrasonic visibility of the shaft 3 are also within the scope of the present invention. An ultrasonic filler material can, for example, be mixed with the polymer overlay coating 67 that is applied to the shaft 3 surface. Alternatively, the shaft 3 may be embedded with microsphere air particles or other ultrasonically visible substance(s) to enhance echogenicity.

Thus in another aspect of the invention, a reinforced fiber shaft is provided that features enhanced visibility under ultrasound and additional damage protection to the core fiber shaft 3. The reinforcement members 11 are comprised of echogenic materials to allow the physician to visualize the entire fiber shaft 3 using an ultrasound probe. The injection of tumescent anesthesia, which is discussed in more detail below, is facilitated by the reinforced shaft 3, which provides a visible target for needle placement. In addition, the reinforcement members 11 provide additional protection by creating a reinforced barrier to prevent damage to the energy-transmitting core 6 of the fiber. This damage can result from unintentional flexing of the fiber shaft 3 as well as from inadvertent needle sticks contacting the fiber 6.

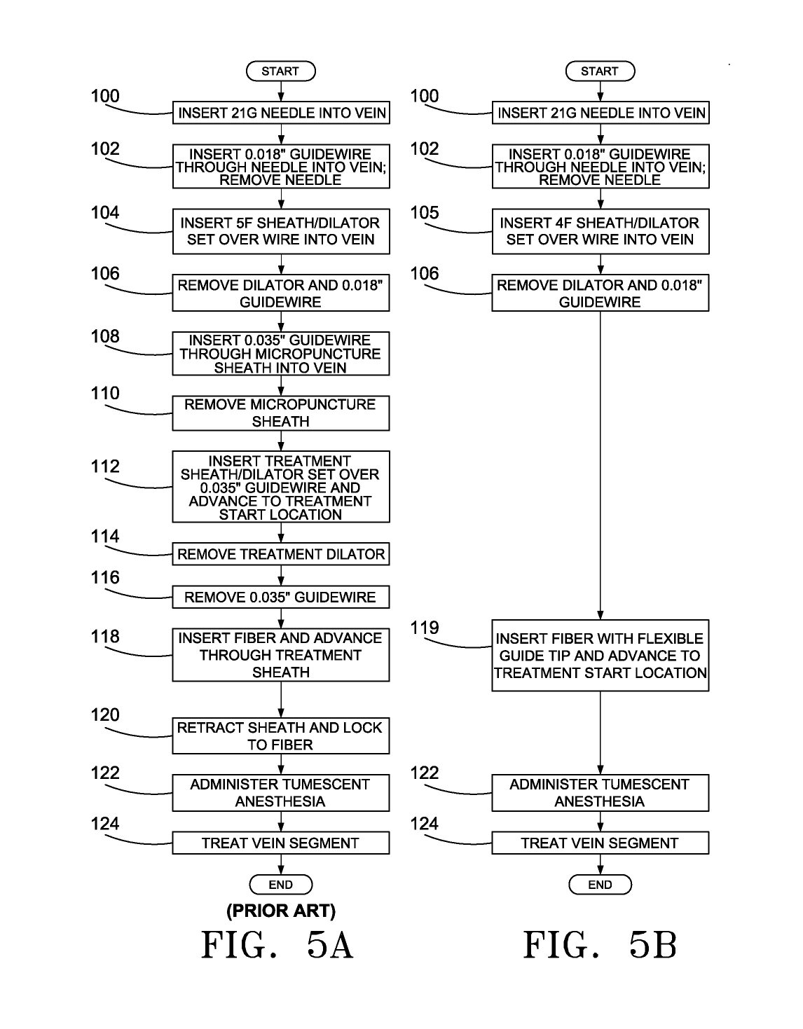

A preferred method of using the endovascular laser fiber with flexible guide tip 1 of the present invention for treating varicose veins will now be described with reference to the flowcharts in FIGS. 5A and 5B. FIG. 5A illustrates the procedural steps of a prior art method of thermally treating varicose veins. FIG. 5B depicts the procedural steps associated with a currently preferred embodiment of the method of the present invention. To begin the procedure, the target vein is accessed using a standard Seldinger technique. Under ultrasonic guidance, a small gauge needle is used to puncture the skin and access the vein (100). An 0.018″ guide wire is advanced into the vein through the lumen of the needle. The needle is then removed leaving the guidewire in place (102). These steps are identical for the prior art method and the improved method disclosed herein.

A micropuncture sheath/dilator assembly is then introduced into the vein over the guidewire (104, 105), after which the dilator and 0.018″ wire are removed, leaving only the sheath in place within the vein (106). With the prior art method, a 5F sheath/dilator assembly is typically required in order to provide sufficient dilation of the entry site to accommodate the subsequent introduction of 6F or larger procedure sheath. With the method of the present invention, on the other hand, a procedure sheath is not required. Accordingly, the insertion site does not require dilation larger than the diameter of a 4F sheath. Thus, the size of the micropuncture sheath/dilator assembly can be smaller and the resulting access site puncture can be reduced relative to prior art methods. As is well-known in the art, smaller access sites are desirable as evidenced by lower patient complication rates including hematoma, bleeding and infection.

Using the method of the present invention, the fiber with guide tip 1 is next inserted directly into the vein through the 4F micropuncture sheath and advanced to a treatment start location without the use of a treatment sheath (119). The fiber is advanced forward through the vessel using the flexible guide tip 9 to facilitate advancement and tracking through even tortuous vessels. Because the fiber with flexible guide tip can easily track through the vessel without accessory components, numerous prior art procedure steps may be eliminated. For example, with the prior art method of use, a 0.035″ guidewire must be first inserted and advanced through the vessel (108), after which the 5F micropuncture sheath is removed (110). In prior art methods, the 0.035″ guidewire is necessary in order to insert and advance the treatment sheath, which is typically a 6F or larger size sheath, to a treatment start location (112). Before inserting the fiber, the dilator and guidewire are removed (114, 116). As shown in FIG. 5A, as compared with the inventive method depicted in FIG. 5B, a total of five additional steps (108, 110, 112, 114, 116) are required with the prior art procedure before the fiber can be inserted and advanced through the diseased vessel (118).

With the prior art method, an additional step is also required to retract the sheath and expose the fiber tip (120). A fiber connector or other mechanism is used to lock the fiber's position relative to the procedure sheath. Locking the two components together is necessary to ensure the relative position of the fiber tip and sheath. Misalignment of the fiber tip can result in thermal energy being transferred to the sheath tip, resulting in potential damage to the sheath and/or patient complications. With the improved method of the current invention, the fiber is positioned relative to the sapheno-femoral junction or other reflux point and the additional step of aligning the fiber tip with the sheath tip encountered in the prior art is eliminated as well as potential damage to the sheath.

In the currently preferred embodiment of the present invention, once the fiber is correctly positioned within the target vessel, tumescent anesthesia is administered along the entire vein segment being treated. Tumescent fluid is injected into the peri-venous sheath surrounding the vein (122), and/or is injected into the tissue adjacent to the vein, in an amount sufficient to provide the desired anesthetic effect and to thermally insulate the treated vein. In one aspect of the invention, the physician uses the reinforced fiber shaft 3, which is highly visible under ultrasound, to guide the injections. The reinforced fiber shaft 3 not only provides a target for injection along the entire treatment segment, but also provides an additional protective barrier to the relatively fragile fiber that minimizes damage that can be caused by inadvertent needle sticks to the shaft during the injection step.

Once the vein has been sufficiently anesthetized, laser energy is applied to the interior of the diseased vein segment. Prior to applying laser energy, the 4F micropuncture sheath may be removed from the vein if desired. For both the prior art method and the current method, the laser generator (not shown) is activated, and the device is withdrawn through the vein segment, preferably at a rate of about 2-3 millimeters per second (124). The laser energy produces localized thermal injury to the endothelium and vein wall causing occlusion of the vein. The laser energy travels down the laser fiber shaft through the energy-emitting face of the laser fiber shaft and into the vein lumen, where the laser energy is absorbed by the blood and, in turn, converted to thermal energy to substantially uniformly heat the vein wall along a 360 degree circumference, thus damaging vein wall tissue, causing cell necrosis, and ultimately causing collapse of the vessel.

The process of controlling the device's pull back speed through the vessel in the case of the prior art method is typically controlled by the use of graduated markings on the procedural sheath. Since a procedural sheath is not used with the improved method, the physician's pullback speed can be controlled by either markings positioned along the fiber shaft or by using an automated pullback mechanism.

The procedure for treating the varicose vein is considered to be complete when the desired length of the target vein has been exposed to laser energy. Normally, the laser generator is turned off when the fiber tip is approximately 3 centimeters from the access site. The physician can monitor the location of the fiber tip relative to the puncture site by the presence of distinguishing marks on the distal segment of the fiber shaft. Once the unique marks appear at the skin surface, the generator is turned off and the laser fiber can then be removed from the body.

In FIGS. 6A, 6B, 7A and 7B, alternative embodiments of the endovascular laser fiber with flexible guide tip 1 of the present invention are illustrated. These embodiments are substantially similar to the embodiments described above with reference to FIGS. 1 through 5, and therefore like reference numerals are used to indicate like elements. The primary difference of the embodiments of FIGS. 6A and 6B in comparison to the embodiments described above is in the construction of the shield 5. In the embodiment of FIGS. 6A and 6B, the shield 5 defines a plurality of thin, axially-extending ribs 90, with circumferentially arranged apertures formed therebetween and defining the windows 7. In the illustrated embodiment, the shield 5 defines four ribs 90 with four apertures extending between the ribs and forming the plurality of windows 7. FIG. 7B illustrates an alternative embodiment of the shield 5. In this embodiment a plurality of thin, helically formed ribs 90 define a plurality of helically shaped windows 7 in shield 5. The shield can be formed from any medical grade metal including nitinol. Although the shield 5 of FIG. 7B includes helically shaped ribs, other rib and window shapes, including other curvilinear shaped ribs, and different numbers of ribs and/or windows than that shown, equally can be employed.

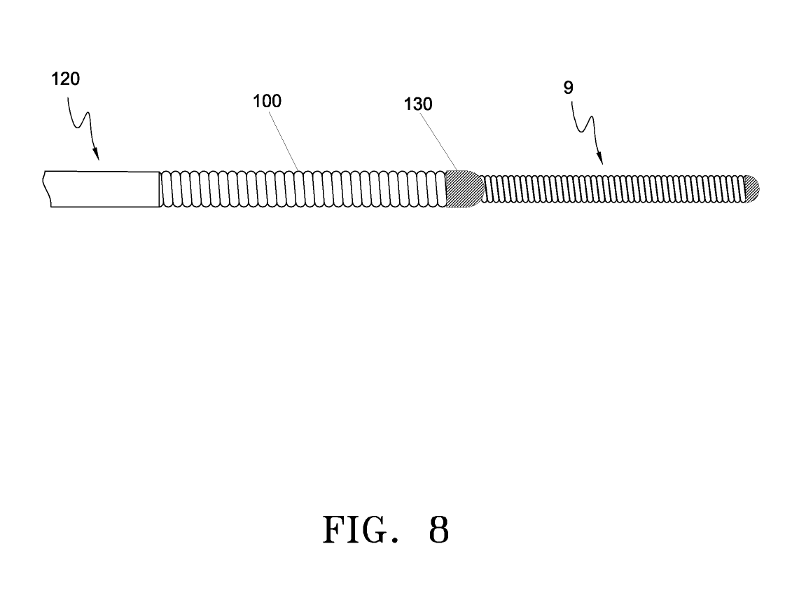

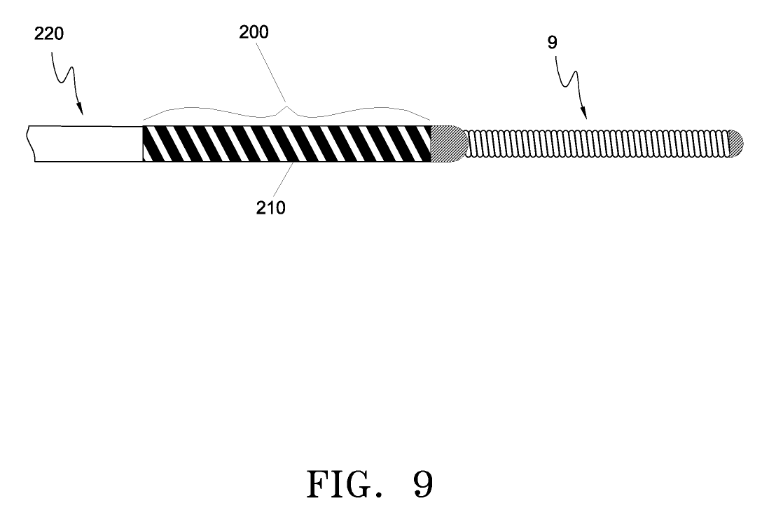

Although the device and method described herein focus on endovenous treatment using laser energy, other thermal energy forms may be used. For example, in one such alternative embodiment as shown in FIG. 8, the energy application device 120 includes one or more RF coils 100 or other electrodes for emission of RF energy located on a distal portion thereof, and the flexible guide tip 9 of the invention extends distally therefrom. The device is comprised of a core made of stainless steel or other conducting material coaxially surrounded by an insulating layer, such as a Teflon® polymer layer. In FIG. 9, microwave antenna 200 includes a flat conductive wire 210 wound in a spiral pattern around the distal portion of the device over the insulating layer for transmitting microwave energy in a radial direction within the vessel. A coating can be applied coaxially over the antenna 200 to create a smooth outer surface and to provide protection to the antenna coil. Optionally, the stainless steel core can extend distally past the antenna to form the mandrel wire of the flexible guide tip. As may be recognized by those of ordinary skill in the pertinent art, blood vessels other than the great saphenous vein and other hollow anatomical structures can be treated using the device and/or methods of the invention disclosed herein.

The invention disclosed herein has numerous advantages over prior art treatment devices and methods. The flexible guide tip can eliminate multiple procedure steps required in prior art methods. Accessory components necessary to complete the prior art procedure steps also can be eliminated, thus enabling a reduction in overall cost of the device. Since the procedure is simplified, there is less time required by the physician to perform the procedure. The leading flexible guide tip not only provides a mechanism for easily tracking and advancing the fiber through even tortuous anatomy, but also facilitates the alignment of the fiber emitting face relative to the source of reflux if desired. Another advantage of the device and method of the currently preferred embodiments of the present invention is the reinforced, ultrasonically visible fiber shaft which provides an easy target for injection of tumescent anesthesia in addition to protecting the fiber core from damage.

The above disclosure is intended to be illustrative and not exhaustive. This description will suggest many modifications, variations, and alternatives that may be made by those of ordinary skill in this art without departing from the scope of the invention. Those familiar with the art may recognize other equivalents to the specific embodiments described herein. Accordingly, the scope of the invention is not limited to the foregoing specification.

What is claimed is:

1. An endovascular thermal treatment device comprising:
an elongated thermal energy delivery device having at its distal portion an energy emitting section; and
a flexible guide tip attached to the distal portion of the energy delivery device and extending distally therefrom, the flexible guide tip adapted to guide the energy emitting section through a blood vessel.

2. The device as defined in claim 1, wherein the flexible guide tip includes a guidewire tip.

3. The device as defined in claim 2, wherein the guidewire tip includes a spring.

4. The device as defined in claim 1, wherein the flexible guide tip includes a coil spring and a rounded portion located distally of the coil spring.

5. The device as defined in claim 1, wherein:
the elongated thermal energy delivery device is an optical fiber having a core and a cladding layer surrounding the core;
the flexible guide tip includes a rounded portion at its distal end; and
the rounded portion is more ultrasonically visible than the optical fiber.

6. The device as defined in claim 1, wherein the flexible guide tip includes a coil spring and a rounded portion located distally of the coil spring, and the rounded portion is more ultrasonically visible than the coil spring.

7. The device as defined in claim 1, wherein the energy delivery device includes an optical fiber and the distal end of the optical fiber defines the energy emitting section.

8. The device as defined in claim 1, wherein the energy emitting section includes at least one radiofrequency electrode.

9. The device as defined in claim 8, further comprising a substantially non-conductive spacer located between the at least one radiofrequency electrode and the flexible guide tip.

10. The device as defined in claim 1, wherein the energy emitting section includes at least one microwave antenna.

11. The device as defined in claim 1, wherein the elongated thermal energy delivery device further includes a shield disposed annularly about the energy emitting section and extending distally therefrom.

12. The device as defined in claim 11, wherein the shield includes at least one window for permitting the flow of blood therethrough.

13. The device as defined in claim 12, wherein the at least one window is a helically shaped window.

14. The device as defined in claim 11, wherein the shield includes a plurality of circumferentially arranged windows for permitting the flow of blood therethrough.

15. The device as defined in claim 1, wherein:
the flexible guide tip includes a rounded portion at its distal end;
the energy emitting section is longitudinally spaced from the rounded portion such that when the rounded portion of the flexible guide tip is located approximately at a sapheno-femoral junction, the energy emitting section is located approximately at a desired start position for treatment.

16. An endovascular thermal treatment device comprising:
an elongated optical fiber having at its distal end an energy emitting face for emitting laser energy;
a flexible guide tip attached to a distal portion of the optical fiber and adapted to guide the energy emitting face through a blood vessel, the guide tip extending distally from the distal portion of the optical fiber.

17. The device as defined in claim 16, wherein the optical fiber further includes a shield disposed annularly about the energy emitting face and extending distally therefrom.

18. The device as defined in claim 17, wherein:
optical fiber includes a core and a cladding layer surrounding the core; and
the shield is more ultrasonically visible than the optical fiber.

19. The device as defined in claim 17, wherein the shield includes at least one window for permitting the flow of blood therethrough.

20. The device as defined in claim 16, wherein the shield includes a plurality of circumferentially arranged windows for permitting the flow of blood therethrough.

21. The device as defined in claim 16, wherein the optical fiber further includes a shield disposed annularly about the energy emitting face and extending both distally and proximally therefrom so as to prevent the energy emitting face from contacting the vessel wall.

22. The device as defined in claim 17, wherein the flexible guide tip is attached to a distal portion of the shield.

23. The device as defined in claim 16, further comprising a reinforcement overlay disposed annularly about the elongated optical fiber to provide an enhanced ultrasonic visibility and structural reinforcement.

24. An endovascular treatment method for causing closure or reducing the diameter of a blood vessel comprising:
advancing through a blood vessel an elongated thermal energy delivery device having at its distal portion an energy emitting section, the distal portion being attached to a flexible guide tip that extends distally from the distal portion;
applying thermal energy through the energy emitting section while longitudinally moving the advanced energy delivery device.

25. The method according to claim 24, wherein:
the energy delivery device includes an optical fiber and the distal end of the optical fiber defines an energy emitting face; and
the step of applying thermal energy includes applying the thermal energy through the energy emitting face.

26. The method according to claim 24, wherein:
the energy delivery device includes an optical fiber and the distal end of the optical fiber defines an energy emitting face;
the optical fiber further includes a shield positioned annularly about the energy emitting face and extending distally therefrom; and
the step of applying thermal energy includes applying the thermal energy through the energy emitting face to heat the blood.

27. The method according to claim 24, wherein:
the energy delivery device includes an optical fiber and the distal end of the optical fiber defines an energy emitting face;
the optical fiber further includes a shield positioned annularly about the energy emitting face and extending distally therefrom;
the shield includes at least one window for permitting the flow of blood therethrough; and
the step of applying thermal energy includes applying the thermal energy through the energy emitting face to heat the blood flowing through the window.

28. The method according to claim 24, wherein the step of advancing includes advancing into the blood vessel the elongated thermal energy delivery device without the use of a treatment sheath.

29. The method according to claim 24, further comprising the step of positioning the elongated thermal energy delivery device so that when the distal end of the flexible guide tip is located approximately at a sapheno-femoral junction, the energy emitting section is located approximately at a desired start position for treatment.

30. The method according to claim 24, wherein:
the energy emitting section includes at least one radiofrequency electrode; and
the step of applying thermal energy includes applying thermal energy through the radio frequency electrode.

31. The method according to claim 24, wherein:
the energy emitting section includes at least one microwave antenna; and
the step of applying thermal energy includes applying thermal energy through the microwave antenna.

32. A method of placing a thermal energy delivery device in a blood vessel comprising:
creating an access site of a blood vessel; and
through the access site, inserting an elongated thermal energy delivery device into the blood vessel without the use of a treatment sheath, the elongated thermal energy delivery device having at its distal portion an energy emitting section, the distal portion being attached to a flexible guide tip that extends distally from the distal portion.

33. The method according to claim 32, wherein:
the energy delivery device includes an optical fiber and the distal end of the optical fiber defines an energy emitting face;
the optical fiber further includes a shield positioned annularly about the energy emitting face and extending distally therefrom;
the shield includes at least one window for permitting the flow of blood therethrough; and
the method further comprises applying thermal energy includes applying the thermal energy through the energy emitting face to heat the blood flowing through the window.

34. The method according to claim 32, further comprising the step of positioning the elongated thermal energy delivery device so that when the distal end of the flexible guide tip is located approximately at a sapheno-femoral junction, the energy emitting section is located approximately at a desired start position for treatment.
